(19) United States
(12) Patent Application Publication
(10) Pub. No.: US 2010/0311482 A1
(43) Pub. Date: Dec. 9, 2010

(54) Electro-Biometric Methods and Apparatus

(76) Inventor: Daniel H. Lange, Kfar Vradim (IL)

Correspondence Address: CARR & FERRELL LLP, 120 CONSTITUTION DRIVE, MENLO PARK, CA, 94025, US

(21) Appl. No.: 12/790,798

(22) Filed: May 29, 2010

Related U.S. Application Data
(60) Provisional application No. 61/182,708, filed on May 30, 2009.

(51) Int. Cl. A63F 13/00; G06F 7/04; A61B 5/04

(52) U.S. Cl. 463/1; 340/5.82; 600/509

(57) ABSTRACT

A method and apparatus for tracking changes in an electro-cardiologic signal using a signal processing device having a sensor. The method comprises communicatively coupling the sensor of the signal processing device with a subject; reading into a memory an electro-cardiologic signal of the subject via the sensor and the signal processing device to create an enrolled electro-cardiologic signal; tracking, via the signal processing device, possible variations in the electro-cardiologic signal of the subject over a time period to modify the enrolled electro-cardiologic signal; and adjusting the enrolled electro-cardiologic signal when changes in the electro-cardiologic signal occur.

### CROSS-REFERENCES TO RELATED APPLICATIONS

This application is related to and claims the priority benefit of U.S. provisional patent application No. 61/182,708, filed May 30, 2009 and titled Electro-biometric Methods and Apparatus. This application is related to U.S. Pat. No. 7,171,680, filed Jul. 29, 2005 (PCT filed Jul. 24, 2003) and titled Method and Apparatus for Electro-biometric Identity Recognition. This application is related to U.S. Pat. No. 7,689,833, filed Nov. 9, 2004 and titled Method and Apparatus for Electro-biometric Identity Recognition. The disclosures of the aforementioned application and patents are incorporated herein by reference.

### BACKGROUND

Identity recognition plays an important role in numerous facets of life, including automatic banking services, e-commerce, e-banking, e-investing, e-data protection, remote access to resources, e-transactions, work security, anti-theft devices, criminologic identification, secure entry, and entry registration in the workplace.

Often computerized systems use passwords and personal identification numbers (PIN) for user recognition. But to maintain security, passwords have to be changed on a regular basis, imposing a substantial burden on the users. Likewise, signature verification methods suffer from other shortcomings, including forgery and enrollment fraud. See for example, U.S. Pat. No. 5,892,824 issued to Beatson et al.

As a result, identity recognition systems that use measures of an individual's biological phenomena—biometrics—have grown in recent years. Utilized alone or integrated with other technologies such as smart cards, encryption keys, and digital signatures, biometrics are expected to pervade nearly all aspects of the economy and our daily lives.

Several advanced technologies have been developed for biometric identification, including fingerprint recognition, retina and iris recognition, face recognition, and voice recognition. For example, Shockley et al., U.S. Pat. No. 5,534,855, generally describes using biometric data, such as fingerprints, to authorize computer access for individuals. Scheidt et al., U.S. Pat. No. 6,490,680, describes identity authentication using biometric data. Dulude et al., U.S. Pat. No. 6,310,966, describes the use of fingerprints, hand geometry, iris and retina scans, and speech patterns as part of a biometric authentication certificate. Murakami et al., U.S. Pat. No. 6,483,929, generally describes “physiological and histological markers,” including infra-red radiation, for biometric authentication. However, these types of technologies have penetrated only limited markets due to complicated and unfriendly acquisition modalities, sensitivity to environmental parameters (such as lighting conditions and background noise), and high cost. In addition, due to complicated acquisition procedures, the foregoing technologies usually require operator attendance.

Fingerprint recognition is well-established and the most mature technology of the group. But it has several drawbacks: a fingerprint recognition system cannot verify physical presence of the fingerprint owner and therefore is prone to deception, limiting its suitability for on-line applications; the optical sensor is a costly and fragile device generally unsuitable for consumer markets; and the system suffers from negative connotations related to criminology.

Retina scanning technologies are characterized by high performance. However, they require high-precision optical sensors, and are not user friendly because they require manipulation of head posture and operate on a very sensitive organ—the human eye. The optical sensor is also costly and fragile.

Iris and face recognition systems are user-friendly technologies since they record an image from afar and are not intrusive. However, they require digital photographic equipment and are sensitive to lighting conditions, pupil size variations and facial expressions. In addition, iris recognition performance is degraded by the use of dark glasses and contact lens, and face recognition may be deceived by impersonation.

Voice recognition is the most user-friendly technology of the group; however, it requires a low-noise setting and is highly sensitive to intrinsically variable speech parameters, including intonation. Moreover, existing conventional recording technologies may be used to deceive speech-based recognition systems.

Thus, a need exists for reliable, robust, hard to deceive (on-line and off-line), low cost, user friendly identity recognition technologies that may be used in stand-alone applications or integrated with existing security systems.

Over the years, electrocardiogram (“ECG”) measurements have been used for many different purposes. ECG signals are electric signals generated by the heart and can be picked up using conventional surface electrodes, usually mounted on the subject's chest. ECG signals are made up of several components representative of different functional stages during each heart beat and projected according to the electric orientation of the generating tissues.

Individuals present different, subject-specific detail in their electro-cardiologic signals due to normal variations in the heart tissue structure, heart orientation, and electrical tissue orientation, all of which affect the electro-cardiologic signals measured from the limbs. Numerous types of systems make use of these subject-specific variations.

For example, Blazey et al., U.S. Pat. No. 6,293,904, describes the use of ECG signals to evaluate or profile an individual's physiological and cognitive state. As to identification, a 2001 conference paper at the 23rd Annual International IEEE Conference on Engineering in Medicine and Biology Society (in Istanbul, Turkey) by Kyoso et al., entitled “Development of an ECG Identification System,” compares a patient's ECG with previously registered ECG feature parameters for purposes of identification. Wiederhold, U.S. Application No. 2003013509, suggests using directly or remotely acquired ECG signals to identify a subject, “explores” feature extraction for identifying individuals, and provides a “preliminary analysis” of such methods.

But an ECG signal is comprised of ECG components having features that may be common to a group. None of these references describe a system or method that eliminates common features of ECG components to create a signature for subject identification. Thus, there still exists a need for systems and methods with these attributes to identify an individual.

The inclusion of the foregoing references in this Background is not an admission that they are prior art or analogous art with respect to the inventions disclosed herein. All references in this Background section are, however, hereby incorporated by reference as though fully set out herein.

### SUMMARY

Applicant provides solutions to the foregoing problems of biometric identification with various apparatuses and methods having several aspects.

In a first aspect, applicant solves each of the foregoing problems of biometric identification through the use of the following method and variations thereof:

producing and storing a first biometric signature that identifies a specific individual by forming the difference between a representation of the heartbeat pattern of the specific individual and a stored representation of common features of heartbeat patterns of a plurality of individuals;

after the producing step, obtaining a representation of the heartbeat pattern of a selected individual and producing a second biometric signature by forming the difference between the heartbeat pattern of the selected individual and the stored representation of the common features of the heartbeat patterns of the plurality of individuals; and

comparing the second biometric signature with the first biometric signature to determine whether the selected individual is the specific individual.

A system, according to this aspect, comprises an ECG signal acquisition module, an ECG signal processing module that comprises an ECG signature generator, and an output module.

Thus, according to this first aspect, the systems and methods disclosed herein transform bio-electric signals into unique electro-biometric signatures. The uniqueness of the electro-cardiologic signatures makes the system very difficult to deceive, and the method's inherent robustness makes it ideal for local as well as for remote and on-line applications. In addition, a biometric-signature-based system is characterized by high recognition performance and supports both open and closed search modes.

In one preferred method according to the first aspect, the stored representation of common features of one or more ECG components is obtained by measuring and storing such representations for a plurality of individuals and then averaging all of the stored representations. Alternately, the common features may be obtained through techniques such as principal component analysis, fuzzy clustering analysis, wavelet decomposition, and the like.

Since electro-cardiologic methods according to this first aspect are robust, they have another important advantage: they permit a simple and straightforward acquisition technology that can be implemented as a low-cost, user friendly acquisition apparatus and also eliminate the need for a skilled operator.

According to a variation on these systems and methods, the common features of one or more of a subject's ECG components may be removed using an analytical model of common features of one or more ECG components, instead of, or in addition to, use of an empirical model. Likewise, the common features may be removed by first classifying the stored representations into subgroups, identifying the common features in at least one subgroup, classifying a subject signal according to subgroup, creating a subject signature by removing the common features of one or more of the subgroup's ECG components from the subject signal, and identifying the subject by calculating the subject signature correlations relative to that subgroup's signatures.

Common features may be determined by averaging synchronized electrocardiograms from a group of individuals and then subtracted from the subject's electrocardiogram to determine the subject's signature. But this method assumes that common features are constant across a group of individuals. In reality, certain common features are present to a greater or lesser degree in any given individual. Therefore, it is better to approximate common features so they make the best fit with a given subject's electrocardiogram before removing them to obtain the subject's signature. This technique provides for a more accurate determination of the subject's signature.

According to this method, a group of electrocardiograms may be broken down (decomposed) into a set of characteristic waveforms. The characteristic waveforms that represent common features of the group are then weighted to best approximate the extent of common features present in the subject's electrocardiogram. The approximation is then subtracted from the subject's electrocardiogram. What remains includes the subject's electrocardiogram signature.

Multiple templates may also be kept for each subject, such as by storing multiple signatures produced by an individual at different pulse rates. In this embodiment, the subject signature may then be correlated with the appropriate template, such as the one for the appropriate pulse rate. Thus, in a variation, the systems and methods disclosed herein may use multiple signature templates to identify an individual over a range of circumstances and reactions. Alternatively, or in addition, according to the first aspect, the subject signal and the enrolled signals may also be normalized based on pulse rate.

According to a second aspect disclosed herein, a process for identification may set a dynamic threshold. This dynamic threshold may be based on a desired level of confidence in the identification, such as one determined by a confidence score.

According to a third aspect disclosed herein, the systems and methods disclosed herein may employ a “Q-factor” to determine whether to reduce signal contamination due to noise. Likewise, the Q-factor or other quality of signal measurement may be used to determine the length of the subject sample required to identify a subject with a desired level of confidence. It may also be used to enroll a sample with the desired level of confidence so that the sample may be suitable for the future comparison.

In an alternate embodiment to the “Q-factor” calculation, the systems and methods disclosed herein may calculate standard deviations in the subject signature and/or enrolled signatures due to noise, and from those calculations determine whether signal quality is appropriate for identification.

Likewise, the systems and methods disclosed herein may determine the signal quality by measuring the impedance of the contact or probe. Signal quality measurements according to this aspect may also be used to inform the subject to adjust his or her contact with or position relative to the sensor or probe.

According to a fourth aspect, the subject and database signatures may be encrypted as a safety precaution against unauthorized access to and use of the signatures.

According to a fifth aspect, the ECG signal may be acquired with electrodes placed in contact with certain body sites that yield a consistent signal. For certain body locations even a slight change of electrode placement may cause drastic changes in the received signal morphology, and may even cause distinct signal components to appear or disappear. Thus, according to this aspect, the methods and systems disclosed herein may use electrode placement sites that produce subject-specific, consistent signals, that are robust notwithstanding changes of electrode placement within the sites. These sites include the arms and legs (including fingers and toes). The robustness of electrode placement within these sites stems from a constant electro-cardiologic signal projection which does not change as long as the electrodes remain close to a limb extremity.

According to this same fifth aspect, certain sensing probes, known as ultra-high impedance sensing probes, may also be used to acquire a signal, including a signal from a single body point such as a fingertip. Alternately, or in addition, these ultra-high impedance probes may remotely sense the electro-cardiologic signal and thereby eliminate the difficulty of electrode placement while maintaining signal consistency.

According to a sixth aspect, the systems and methods disclosed herein may comprise elements and steps that protect against enrollment fraud and reduce the ability of a database enrollee to misrepresent his or her identity.

According to a seventh aspect, the systems and methods disclosed herein may identify a subject by comparing his or her match scores with the match scores of database enrollees.

According to an eighth aspect, the systems and methods disclosed herein may use weighted correlation techniques, ascribing different weights to different electro-cardiologic signal components for the purpose of producing a signature. Alternatively, or in addition, signatures may be normalized using a variety of metrics including root-mean-square computations or L1 metrics.

Some biometric technologies employ challenge-response protocols to ensure that the user data that they receive is live. In that way, they can reduce the risk that the system can be spoofed by the playback of biometric data. But, to date, the challenge-response mechanisms for biometric systems have required active participation by the user. And active user participation complicates and extends the user verification process. For example, speech recognition systems typically require the user to repeat a randomly selected word or sentence. Therefore, according to another aspect, a biometric ID system may reduce the risk of spoofing by beneficially employing a biological-challenge-response mechanism that does not require a conscious response from the user.

The systems and methods according to each of the foregoing aspects preferably perform their tasks automatically for the purpose of identity recognition. Further, these systems and methods can be incorporated into a wide range of devices and systems. A few non-limiting examples are as follows: a smart card; a passport; a driver's license apparatus; a Bio-logon identification apparatus; a personal digital assistant (“PDA”); a cellular-embedded identification apparatus; an anti-theft apparatus; an ECG monitoring apparatus; an e-banking apparatus; an e-transaction apparatus; a pet identification apparatus; a physical access apparatus; a logical access apparatus; and an apparatus combining ECG and fingerprint monitoring, blood pressure monitoring and/or any other form of biometric device.

Further, the systems and methods disclosed herein can be used to identify a person's age, such as by comparing the width of a subject's QRS complex, or more generally the subject's QRS-related signature component, with those of an enrolled group or analytical ECG model.

In another application, the systems and methods herein may be used to identify persons on medication, such as by enrolling and calculating, or analytically deriving, a series of drug-related signature templates. This method may also be used to identify or catch subjects who would attempt to fool the system by using medication to alter their ECG signal.

Other applications include using the systems and method disclosed herein for building and room access control, surveillance system access, wireless device access, control and user verification, mobile phone activation, computer access control (including via laptop, PC, mouse, and/or keyboard), data access (such as document control), passenger identification on public transportation, elevator access control, firearm locking, vehicle control systems (including via ignition start and door locks), smart card access control and smart card credit authorization, access to online-line material (including copyright-protected works), electronic ticketing, access and control of nuclear material, robot control, aircraft access and control (passenger identity, flight control, access of maintenance workers), vending machine access and control, laundromat washer/dryer access and control, locker access, childproof locks, television and/or video access control, decryption keys access and use, moneyless slot machines, slot machine maintenance access, game console access (including online transaction capability), computer network security (including network access and control), point-of-sale buyer identification, on-line transactions (including customer identification and account access), cash payment service or wire transfer identification, building maintenance access and control, and implanted medical device programming control. Other applications will be apparent to those skilled in the art and within the scope of this disclosure.

For any application, an apparatus according to any or all of the foregoing aspects can operate continuously or on demand. The apparatus can be constructed to obtain the representation of the heartbeat pattern of a selected individual by having one or more electrodes in contact with individual or sensors remote from the individual. When the apparatus is provided in a smart card, the card can be enabled for a limited period of time after successful recognition and disabled thereafter until the next successful recognition is performed. The apparatus can be constructed to operate with encryption keys or digital signatures.

As to the methods disclosed herein, the steps of the foregoing methods may be performed sequentially or in some other order. The systems and methods disclosed herein may be used on human or other animal subjects.

Each of these aspects may be used in permutation and combination with one another. Further embodiments as well as modifications, variations and enhancements are also described herein.

### BRIEF DESCRIPTION OF THE DRAWINGS

FIG. 1 is a simplified block diagram of a system for use with the aspects disclosed herein composed of a signal acquisition module, a signal processing module, and an output module.

FIG. 2 is a block diagram of an embodiment of the signal acquisition module of the system of FIG. 1.

FIG. 3 is a block diagram of an embodiment of the signal processing module of the system of FIG. 1.

FIG. 4 shows the first six most influential PCs extracted from a pool of one-hundred subjects, and the contribution of the first ten PCs to the representation of data variance.

FIG. 5 shows the original electrocardiographic signals and their respective signatures constructed by eliminating the optimal combination of the three most influential PCs and their latency shifted versions.

FIG. 6 is a diagram showing a grand-average electro-cardiologic signal waveform calculated from a database of 20 subjects.

FIG. 7 shows a group of electro-cardiologic signal waveforms of ten of the subjects participating in the database and contributing to the average waveform of FIG. 4.

FIG. 8 shows a group of electro-biometric signature waveforms, or templates, derived from the signal waveforms of FIG. 7.

FIG. 9 shows a scatter plot and distribution histograms of the sign-maintained squared correlation values of the subjects who contributed to the grand average waveform of FIG. 4.

FIG. 10 shows a table of z-scores based on the desired degree of confidence in the identification cut-off.

FIG. 11a shows a distribution of correlation.

FIG. 11b shows a distribution of Z-transformed correlations.

FIG. 12 shows identification performance curves (static).

FIG. 13 shows identification performance curves (dynamic).

FIG. 14 shows signal quality as a function of NSR.

FIG. 15 shows match score distribution as a function of signal quality for 5 second segments.

FIG. 16 shows match score distribution as a function of signal quality for 20 second segments.

FIG. 17 shows match score as a function of recording duration (for Q=0.8).

FIG. 18 shows match score as a function of recording duration (for Q=0.5).

FIG. 19 shows a functional component diagram of a preferred system.

FIG. 20 shows a functional component diagram of a preferred signal processor.

FIG. 21 shows a screen shot of a preferred embodiment.

FIG. 22 shows a screen shot of a preferred embodiment.

FIG. 23 shows a screen shot of a preferred embodiment.

FIG. 24 shows a screen shot of a preferred embodiment.

FIG. 25 shows a data screen shot of a preferred embodiment.

### DETAILED DESCRIPTION

### Definitions

Unless otherwise indicated, the meaning of the terms “identify,” “identifying” and “identification” include the concepts of “verify identity,” “verifying identity,” and “verification of identity,” respectively.

“Closed search” means a search in which a single stored signature is examined to verify the identity of an individual.

“Open search” means a search in which a plurality of stored signatures are searched to identify a subject.

### First Aspect:

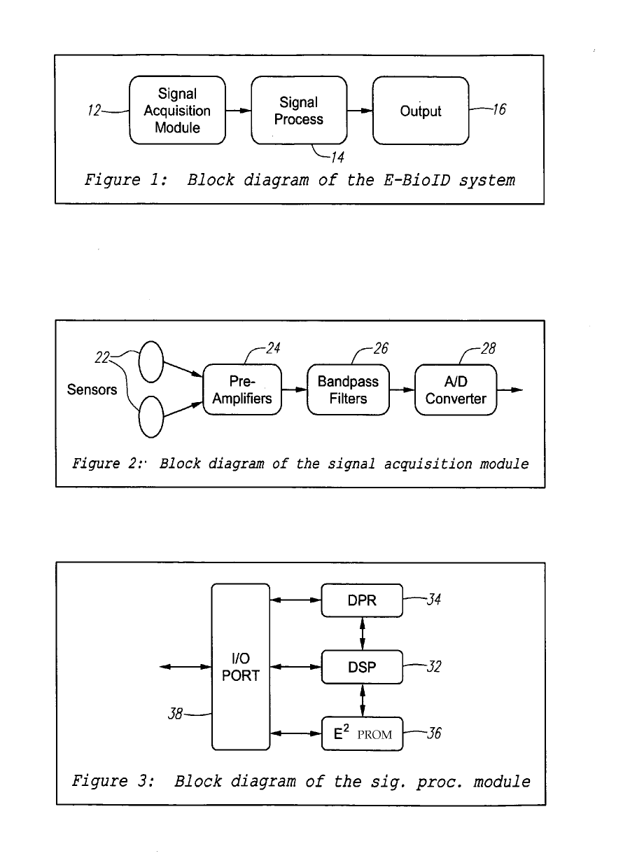

According to the first aspect, a bio-electric signal is acquired, processed and analyzed to identify the identity of an individual. A preferred embodiment of a system and a method according to this first aspect is illustrated, by way of example, in FIG. 1. FIG. 1 shows a system called an Electro-Biometric IDentification (E-BioID) system. In this preferred embodiment, the stored representation of the common features of the one or more ECG components of the plurality of individuals is the average of those individuals' one or more ECG components. However, other embodiments can utilize stored representations of different types of common features, such as those attainable by, for example, principal component analysis, fuzzy clustering analysis, or wavelet decomposition, or provided by an analytical model.

In the preferred embodiment, the basic elements of the E-BioID system include a signal acquisition module 12, a signal processing module 14, and an output module 16, implemented in a single housing. In another preferred embodiment, the system may provide for remote analysis of locally acquired electro-biometric signals. Each of the components shown in FIG. 1 can be readily implemented by those skilled in the art, based on principles and techniques already well known in the art in combination with the present disclosure.

FIG. 2 shows a preferred construction of the signal acquisition module 12 in an E-BioID system. The data acquisition module preferably includes one or more sensors 22, pre-amplifiers 24, band-pass filters 26 and an analog-to-digital (A/D) converter 28. Each of these components can be readily implemented by those skilled in the art, based on principles and techniques already well known in the art in combination with the present disclosure.

Sensors 22 can be of any type capable of detecting the heartbeat pattern. For example, they can be metal plate sensors that are an “add-on” to a standard computer keyboard. According to another aspect, a single sensor may, by itself, acquire the signal from a single point of contact, such as by contacting a finger; alternately, the sensor may not need to touch the subject at all.

FIG. 3 shows preferred elements of signal processing module 14 in the E-BioID system. The signal processing module preferably includes a Digital Signal Processor (DSP) 32, a Dual Port Ram (DPR) 34, an Electrically Erasable Programmable Read Only Memory (E2PROM) 36 and an I/O port 38. Each of these components can be readily implemented by those skilled in the art, based on principles and techniques already well known in the art in combination with the present disclosure. Signal processing module 14 is connected to signal acquisition module 12 and output module 16 via port 38.

In an alternative embodiment, the signal processing module may be implemented, with suitable programming, on a personal computer, which is a flexible computation platform, allowing straight-forward integration of the system into existing computing facilities in a home, office, or institute/enterprise environments.

Output module 16 preferably consists of a dedicated display unit such as an LCD or CRT monitor, and may include a relay for activation of an external electrical apparatus such as a locking mechanism. Alternatively, the output module may include a communication line for relaying the recognition result to a remote site for further action.

### Signal Acquisition, Processing and Analysis

Bioelectric signals, or heartbeat signals, are acquired in a simple manner, where the subject is instructed to touch at least one sensor 22 for a few seconds. The one or more sensors, which may be metal plates, conduct the bioelectric signals to the amplifiers 24, which amplify the bioelectric signals to the desired voltage range. In a preferred embodiment, the voltage range is zero to five volts.

The amplified signals pass through filters 26 to remove contributions outside a preferable frequency range of 4 Hz-40 Hz. Alternatively, a wider range of 0.1 Hz-100 Hz may be used in conjunction with a notch filter to reject mains frequency interference (50/60 Hz). Digitization of the signal is preferably performed with a 12-bit A/D converter 28, at a sampling frequency of preferably about 250 Hz.

In module 14, the signals are normalized by the ‘R’ peak magnitude, to account for signal magnitude variations which mostly relate to exogenic electrical properties. The normalized data is transformed into an electro-biometric signature which is compared to pre-stored electro-biometric signature templates. The result of the comparison is quantified, optionally assigned a confidence value, and then transmitted to output module 16, which provides recognition feedback to the user of the E-BioID system and may also activate external apparatuses such as a lock or siren, virtual apparatuses like network login confirmation, or a communication link.

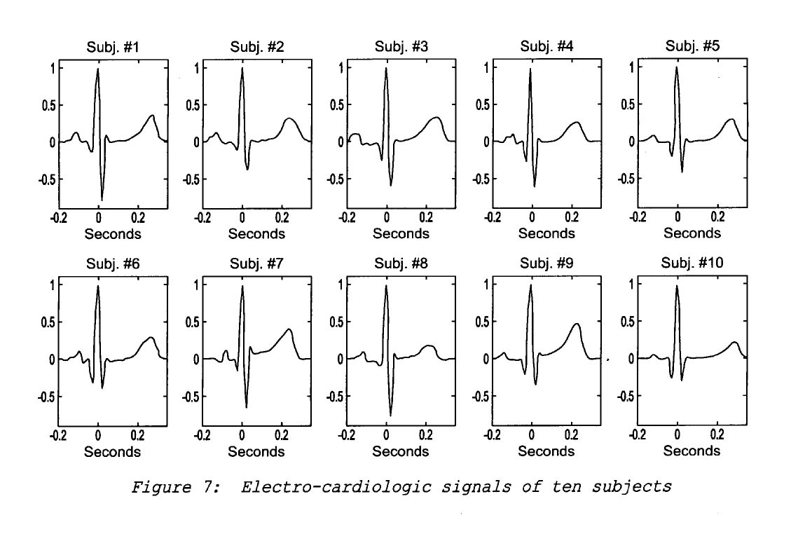

Alternately, or in addition, the signal may be normalized for pulse rate. This is useful because electro-cardiologic signals are affected by changes in pulse rate, which is a well-known electro-cardiologic modifier. Pulse rate changes may cause latency, amplitude and morphological changes of the ‘P’ and ‘T’ components relative to the ‘QRS’ component of the electro-cardiologic signal (these components appear in FIG. 7). However, pulse rate changes may be automatically compensated for by retrospective, pulse rate-driven adjustment of the signal complex. Moreover, an adaptive operation mode of the system can track and compensate for pulse rate induced changes. This can be done by compressing or expanding the time scale of one cycle of the heartbeat waveform. More sophisticated formulations describing the relations between waveform characteristics (e.g. S-T, P-Q segment durations) and pulse rate may be used. Thus, a method according to this variation may be based on electro-cardiologic signal discrimination, wherein analysis is carried out synchronously with the heart beat, eliminating features common to the general population and thus enhancing subject-specific features that constitute an electro-biometric, or biometric, signature, normally undetectable in raw electro-cardiologic signals.

In another embodiment, the E-BioID system is implemented as a fully integrated compact device, where many of the functional elements are implemented on an ASIC based system.

In another embodiment, the apparatus can be incorporated into a watch worn on the wrist, where the signal is measured between the wrist of the hand on which the watch is worn and the other hand of the wearer. The back side of the watch may be made of a conductive medium (e.g. a metal plate) in contact with the back of the wrist, and the face of the watch can be provided with another metal contact that needs to be touched with a finger of the other hand. The watch may transmit a signal indicating confirmation of the identity of its wearer, and/or activating a physically or logically locked device such as a door, a computer, a safe, etc. The watch may also transmit personal information about its wearer. In the same manner, the apparatus can be incorporated into a belt, or any other apparel item comprising a conductive medium. The belt or other apparel item may then transmit a signal indicating confirmation of the identity of its wearer, and/or activating a physically or logically locked device and/or transmitting personal information about its wearer.

### Principle of Operation

Biometric recognition requires comparing a newly acquired biometric signature against signature templates in a registered or enrolled biometric signature template database. This calls for two phases of system operation: Enrollment and Recognition.

### Enrollment Phase

In a preferred embodiment, each new subject is instructed to touch a first sensor with a finger of the left hand, while simultaneously touching another sensor with a finger of the right. In alternative embodiments, the subject may touch the sensors, typically made of metal, with other parts of the body, preferably the hands or legs. In another embodiment, the subject may touch a single sensor with a single body point. Alternately, the subject need not touch a sensor at all. The system monitors the subject's pulse rate and initiates a recording, preferably lasting for at least 20 seconds. Shorter intervals may be used depending on the required level of accuracy. Once the recording is complete, the system may perform a self-test to verify signature consistency by comparison of at least two biometric signatures derived from two parts of the registered segment. The two parts may be two halves, or two larger, overlapping, segments. The two parts may be used to derive two biometric signatures. If the self-test result is successful, enrollment of that subject is complete, and if unsuccessful the procedure is repeated. The successful recording is used for construction of an electro-cardiologic signal or a series of electro-cardiologic signals, which are added to an electro-cardiologic signal database.

The electro-cardiologic signals are then transformed into a set of electro-biometric signature templates by eliminating features that are common to all or a subset of the subjects participating in the dataset, thereby enhancing subject-specific discriminating features.

In a preferred embodiment, the system creates a grand-average electro-cardiologic template, which is calculated by synchronous averaging of normalized electro-cardiologic signals from the entire pool of subjects. The grand-average represents the above-mentioned common features, and thus subtraction of the grand-average from each one of the electro-cardiologic signals yields a set of distinct, subject-specific electro-biometric template signatures. In an alternative embodiment, other means for elimination of the common features may be used, such as a principal component analysis, fuzzy clustering analysis or wavelet decomposition.

In a more preferred embodiment, a group of electrocardiograms may be broken down (decomposed) into set of characteristic waveforms. According to this preferred embodiment, noise is removed from the electrocardiograms of a group of individuals. The system may use Principal Component Analysis (PCA) to decompose the group's electrocardiograms into a set of orthogonal (non-correlated) components. These non-correlated components, taken together, represent the entire energy of the signals—that is 100% of the signal variance.

The first principal components are those associated with largest eigen values of the PCA representation. Usually the first three to five components and, in any event, less than the first ten components of the group's electrocardiograms typically represent approximately 90% of the electrocardiogram's energy or variance and contain the common features. Remarkably, these first components represent common features that are present and stable across the human population at large. As a result, these first principal components can be used to identify the signature of any human subject and need not be recalculated for each subject. The remaining smaller components (which typically can be 10% of the total waveform energy) represent noise and some individual information of the group.

The characteristic waveforms that represent common features of the group are then subtracted from the subject's electrocardiogram. What remains includes the subject's electrocardiogram signature plus some remaining noise.

Characteristic waveforms may be created in different ways, and depend on the desired “distance” or “overlap” between each waveform. For example, the correlation function may preferably be used to determine the desired distance between waveforms, although other methods also work.

Remarkably, if an electrocardiogram is taken from an individual who has not participated in the enrollment data set, it is possible to determine his or her electrocardiogram signature usually with reference to just the first three to four PCA components of the enrolled data set and time shifted versions of them.

Determining the Signature

All subjects' electrocardiograms contain each of the first principal components to greater or lesser degrees. According to this preferred embodiment, a subject's electrocardiogram may be approximated using the principal components from the sample set according to the following equation.

$${\sum }_{i = 1}^{p} {C}_{i} {PC}_{i} = {ECG}_{individual}$$

In this equation, Cᵢ is a reconstruction coefficient, p is the model order and PC is the principal component. The goal is to find the coefficients that weight the database principal components for the best approximation of the subject's electrocardiogram. In other words, the goal is to minimize the error between an approximation of the subject signal constructed by the weighting the database's principal components and the original subject signature.

This may be done by a variety of methods. One method is to determine reconstruction coefficients using a least squares approximation to minimize the norm of the reconstruction error. This is shown below:

$$‖ {ECG}_{ind} - \sum {C}_{i} {PC}_{i} ‖ OR Error = {\sum }_{n = 1}^{N} {( {ECG}_{n} - \sum {C}_{i} \cdot {PC}_{i} )}^{2}$$

Once the optimal coefficients are determined, they may be used to sum the database's first principal components (such as the top 3 or 4) according to the following equation:

$${\sum }_{i = 1}^{3 or 4} {C}_{i} \cdot {PC}_{i} = Sum$$

This sum is then subtracted from the subject signal. What remains is the subject signature and perhaps some noise.

Further, since noise, by definition, is uncorrelated, it is usually described by the last principal components—those that are associated with the smallest eigen values. As a result, noise may be optionally removed from the subject signal by weighting these last principal components to make the optimal fit with the subject signature and then removing them from the subject signal. Noise may also be removed by other methods.

### Accounting for Latency Variation

Some of the variation in an electrocardiogram component database is due to latency changes, namely time variance in enrolled data signatures. As a result, the foregoing method may be enhanced by time shifting the principal components, preferably both to the left and to the right. For example, if three principal components are used to approximate common electrocardiogram features, then six more components could be added to account for latency variation—two for each component, shifted left and shifted right.

In this example, the three principal components and the six time shifted components would be used to calculate the construction coefficients. And once the best construction coefficients are determined, the common feature components are constructed and subtracted from the original subject electrocardiogram signature to yield the individual signature:

$$Signature = {ECG}_{n} - {\sum }_{i = 1}^{P} {C}_{i} \cdot {PC}_{i}$$

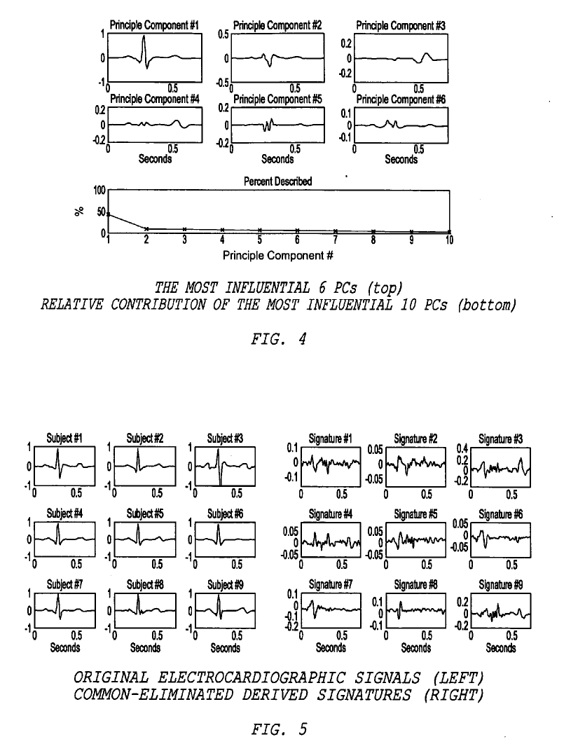

FIG. 4 shows the first six most influential PCs extracted from a pool of one-hundred subjects, and the contribution of the first ten PCs to the representation of data variance. FIG. 5 shows the original electrocardiographic signals and their respective signatures constructed by eliminating the optimal combination of the three most influential PCs and their latency shifted versions.

Although PCA is a robust algorithm that provides a progressive, influential representation of components with clear distinctions in magnitude between the main signal, secondary variations and noise, at least two alternate techniques may be used to decompose the group's electrocardiograms. In a first alternate embodiment, independent component analysis (ICA) may be used to decompose compound signals into independent components (as opposed to the orthogonal components of PCA). These independent components may then be used for modeling and reconstruction of electrocardiograms in a manner similar to PCA.

In a second alternate embodiment, wavelet decomposition (WD) may be used to decompose compound signals into a set of time-scaled waveforms called wavelets. WD is based on a transient wavelet waveforms, as opposed to Fourier decomposition (which is based on continuous sine and cosine decomposition). As a result, WD has an advantage over Fourier analysis in that wavelets are more efficient descriptors of transient signal components such as electrocardiograms.

Alternately, or in addition, common features may be removed by using an analytical model for common features of one or more ECG components rather than by using an empirical model calculated from the enrolled data.

In another preferred embodiment, the database is divided into several subsets in a way that enhances intra-subset similarity and inter-subset disparity. The embodiment then calculates a distinct grand-average or other common feature determination for one or more of the subsets. This database partition itself may be performed using standard pattern classification schemes such as linear classifiers, Bayesian classifiers, fuzzy classifiers, or neural networks. In case of a large database, it is useful to partition the database into subsets in order to simplify and shorten the search process as well as to ensure the validity of the grand-average as an appropriate representative of similarity among the electro-cardiologic signals. The subject signature may then be created by removing common features found in the appropriate subgroup.

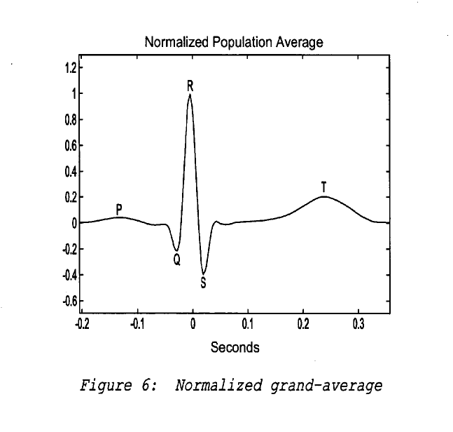

FIG. 6 shows an example of a grand-average, constructed from a pool of 20 subjects participating in the database.

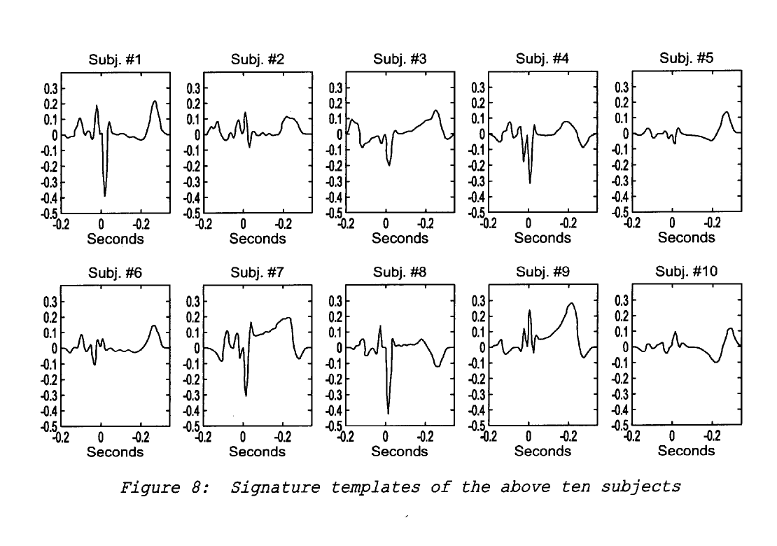

FIG. 7 shows examples of electro-cardiologic signals, and FIG. 8 shows the electro-biometric template signatures derived from the above electro-cardiologic signals by elimination of features common to all the subjects included in the database. Specifically, each signature of FIG. 8 is obtained by subtracting the waveform of FIG. 6 from the corresponding signal of FIG. 7. It will be observed that while the original electro-cardiologic signals are highly similar, the derived electro-biometric signatures are markedly different. These differences have been found to reflect inherently unique electro-cardiologic disparity which underlies the recognition capabilities of the E-BioID system.

### Recognition Phase

In the recognition phase, the subject interacts with the system in a similar manner to that of the enrollment phase, however a shorter recording time on the order of a few seconds is sufficient.

In a preferred embodiment, the system executes a verification procedure (closed search): the system processes the acquired signals, forms an electro-biometric subject signature by removing common features found in the entire database, found in a partitioned subgroup of the database or provided by analytical ECG model, adjusts the signature according to the pulse rate, and compares the adjusted electro-biometric signature with the subject's enrolled electro-biometric signature template.

In another preferred embodiment, the system executes an identification procedure (open search): the system repeats the comparison process for the entire database or a partitioned sub-group of the database, thereby providing identification of the matching identity.

### The Comparison Process

In a preferred embodiment, the comparison is performed by calculation of a correlation coefficient, q, between an electro-biometric signature σⱼ and an electro-biometric signature template Φi, as follows:

$$ρ = \frac{COV ⌊ {σ}_{j} , {Φ}_{i} ⌋}{\sqrt{VAR [ {σ}_{j} ] VAR [ {Φ}_{i} ]}} .$$

The correlation coefficient is squared, maintaining its original sign: η=sign(ρ)*|ρ|². In an alternative embodiment, the comparison may be based on other similarity measures, such as RMS error between the electro-biometric signatures.

The comparison may yield one or several correlation coefficients, depending on the mode of operation: closed search; or open search. In a closed search mode, the sign-maintained squared correlation coefficient (i) is used for making the recognition decision: a value greater than a preset threshold is regarded as a positive identification, or a match; borderline, near-threshold values may indicate a need for extended or repeated recording. In an open search mode, the largest sign-maintained squared correlation coefficient among all sign-maintained squared correlation coefficients yields the most likely subject identification, provided that the highest coefficient is above a selected threshold.

The preset threshold is derived from the required confidence level; higher desired confidence levels require higher thresholds. In one embodiment, sign-maintained squared correlation values larger than 0.8 are characteristic of a match and values lower than 0.7 are characteristic of a mismatch. Thus, sign-maintained squared correlation values higher than 0.8 may be considered as true matches and values lower than 0.7 as mismatches.

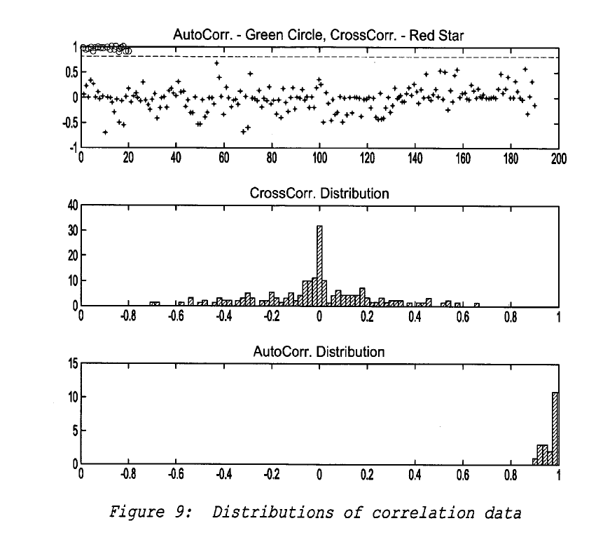

The upper diagrams of FIG. 9 shows a scatter plot of sign-maintained squared correlation values, marking the 0.8 threshold with a dashed line. A clear separation between matches (circles) and mismatches (stars) is evident. The histograms in the other two diagrams provide a different view of the powerful recognition capabilities of the E-BioID system, where it can be seen that the mismatches are concentrated around the zero value (no correlation) while matches are densely distributed near 1.0 (absolute correlation).

In alternative embodiments, more sophisticated decision schemes may be used such as multi-parameter schemes (e.g. fuzzy logic schemes), which use more than one distance measure; for example, multiple correlation values can be derived from segmented data analysis.

In a preferred embodiment, the system improves its performance with time by adding electro-cardiologic signals to the subject's database file when changes in the signals are encountered. In subsequent recognitions, the system processes the newly acquired signals, calculates the pulse rate, forms an electro-biometric subject signature, selects the enrolled electro-biometric signature template with the most similar pulse rate, and compares the new electro-biometric signature with the selected enrolled electro-biometric signature template.

In another preferred embodiment, the system uses signals acquired during long-term system operation to track possible variation in the enrolled subject electro-cardiologic signal and, if consistent changes occur, the enrolled signal is automatically adjusted to reflect these changes. This tracking process compensates for gradual changes in the electro-cardiologic signal over long time periods, but does not compensate for fast, acute changes like those expected in connection with clinical heart conditions. In another embodiment, such acute changes may be reported to the subject indicating a need for medical consultation.

The systems and methods can recognize large, notable changes that should be immediately checked by a physician. Many such changes in heart activity will be simply and easily detectable with this system. The system can also recognize smaller, cumulative heart activity changes over time. Compared to a conventional medical ECG procedure, which compares a current ECG to one that is typically a year old, the system and methods herein, henceforth “Cardio Tracker”, have the advantage of many more readings over time, which will give the system increased confidence in their accuracy. The Cardio Tracker can also analyze ongoing trends, potentially predicting problems before they reach clinical significance. A screen shot of one Cardio Tracker embodiment is shown at FIG. 21.

As described herein, an ECG (electrocardiogram) contains individually unique elements which can be used to identify an individual. In one embodiment of this Cardio Tracker application, elements are extracted by techniques based on principal component analysis to create individualized identification signatures. Identification is achieved using a thresholded correlation-type measure, which relates candidate identification attempts to the stored signature.

These signatures, while stable, will present minute yet unique variation based on the relative physiological health of the user.

The degree of variation can be expressed as a correlation-type score and/or mean-square error relative to the original enrollment template.

Alternative scores can be derived from various transformations of both the original and candidate signature.

The degree of variation provides a direct index metric for deviation from enrolled health status, i.e., the greater the deviation from the original signature, the greater the change in health status.

If the signature is initially taken during a period of relatively good health, or even during pre-existing stable condition, increasingly poor health on later identification attempts will be associated with increasing deviation of certain signature elements from the stored original signature.

Additional information on health relationships can be derived from the pulse rate, and analysis of standard components of the ECG. The combination of signature analysis and pulse/ECG component analysis will provide information not easily derived by either method alone. In particular, assessments of whether the changes are improvements or decrements in health status can be derived from inspection of signature and ECG component modalities.

Changes in signature may also be related to psychological state and mood. Thus in another preferred embodiment, the system, “Cardioscope,” is applied to measure and examine a user's signatures, using an individualized historical database, providing an instant reading of the user's current physiological state. Like a horoscope, the Cardioscope can then predict optimal activities, based on the current signature(s). Unlike horoscopes, however, the signature analysis provides a scientific basis for its predictions. The Cardioscope will ask about success at different activities, ranging from romantic to financial and professional. Statistical analysis will match each signature with its best activity. With repeated use, the system will eventually learn to predict which activity is most appropriate, based on the current signature(s).

In a most preferred embodiment of this Cardioscope application, the elements are extracted by techniques based on principal component analysis to create individualized identification signatures. Identification is achieved using a thresholded correlation-type measure, which relates candidate identification attempts to the stored signature.

These signatures, while stable, present minute yet unique variation based on the psycho-physiological reaction patterns of the user. That is, different states of psycho-physiological preparation for different types of activities will be apparent by analysis of the signatures.

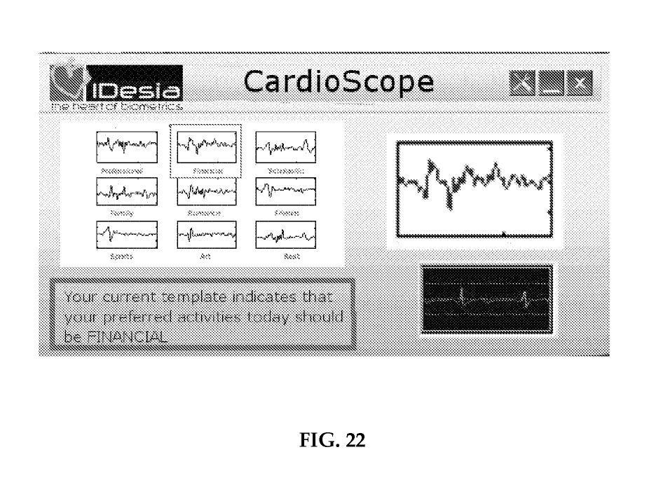

Success at different activities (professional, financial, romantic, etc.) can be associated with the signature variations. Different activities will be associated with different variations. See, for example, FIG. 22, a screen shot of one possible implementation of this embodiment.

Historical associations between success at different activities and different recorded signatures can be acquired by using psychological questionnaire procedures (such as magnitude estimation scores).

In this most preferred embodiment, analysis may rely on correlational relationships between historical successful activities (and their degree of success) and the signatures recorded coincident to these activities.

The degree of correlation between the current signature and the historical signatures yields a predictive index of successful performance of each of the analyzed activities.

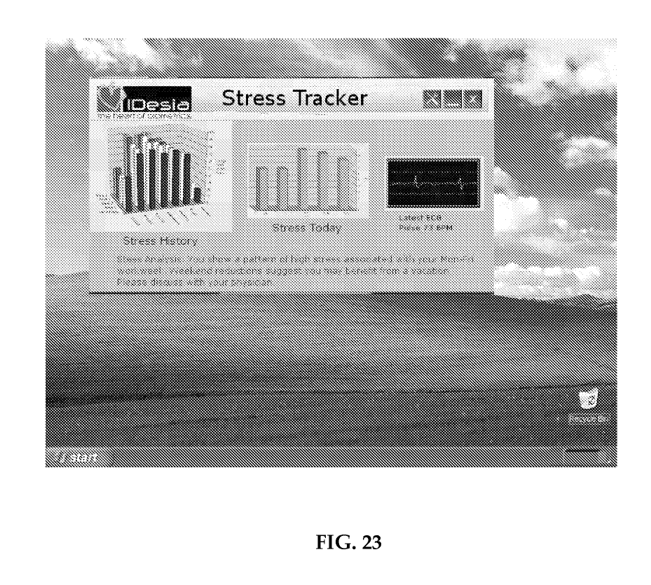

In another application, the cardio-graphic technology disclosed herein may be implemented in a Stress Tracker system that can assess and quantify pulse rate and ECG parameters related to stress. Thus, databases and algorithms can record and analyze stress levels, and determine how they are related to the user's lifestyle. This can be the first step in a program to reduce overall stress levels, with additional training software. Week-by-week stress history, current stress measurements, an ECG sample with pulse, and a computerized analysis all urge the user to take better care of him or herself. As shown in FIG. 23, a stress bar, indicating the stress level at the latest reading, may be visible on the lower right of the Windows task bar.

In a most preferred embodiment of this stress tracking application, These elements are extracted by techniques based on principal component analysis to create individualized identification signatures. Identification is achieved using a thresholded correlation-type measure, which relates candidate identification attempts to the stored signature.

These signatures, while stable, present minute yet unique variations based on the physiological state of the user, in this case, stress. These signature variations can be characterized, classified, and related to stress level, by various means for metric quantification such as mean-square difference.

The classification process can be achieved by a combination of statistical analysis, based on techniques such as principal components analysis, independent components analysis, discriminant analysis, CART (classification and Regression Trees), and/or neural networks.

If the signature is initially taken during a period of relative calm (non-stress), or even during preexisting stress, increasing or decreasing stress on later identification attempts will be associated with increasing deviation of certain signature elements from the stored original signature. These changes in deviation can be modeled and scaled with correlation-based methods, which provides a measurement metric for physiological stress level.

Additional information on stress relations can be derived from the pulse rate, and analysis of standard components of the ECG. The combination of signature template analysis and pulse/ECG component analysis will provide information not easily derived by either method alone.

The stress level determination as utilized in the Stress Tracker can also be used to verify/validate/enhance the efficacy of biofeedback training. Biofeedback is a method used to train individuals to control autonomic functions, such as heart rate/pulse, which are not normally under voluntary control. Pulse rate reduction can be achieved by controlling breathing rate and depth. ECG signatures can be used to refine the biofeedback.

The ECG (electrocardiogram) contains individually unique elements which can be used to identify individual people. In a preferred embodiment of this biofeedback technique, elements are extracted by techniques based on principal component analysis to create individualized identification signatures. Identification is achieved using a thresholded correlation-type measure, which relates candidate identification attempts to the stored signature.

These signatures, while stable, present minute yet unique variations based on the physiological state of the user, in this case, stress. Stress level is generally related to pulse rate—for an individual, the higher the pulse rate, the higher the stress level. As previously described, signature variations can be characterized, classified, and related to stress level. Thus, the stress level determination as utilized in the Stress Tracker can be used to verify/validate/enhance the efficacy of biofeedback training.

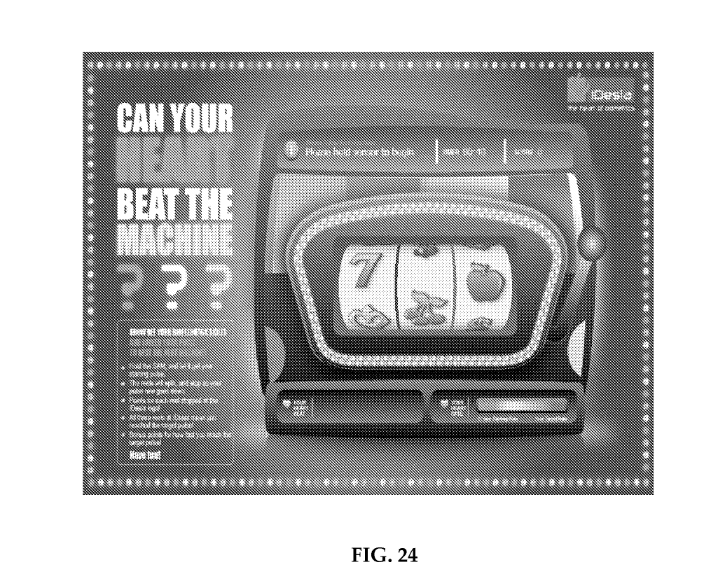

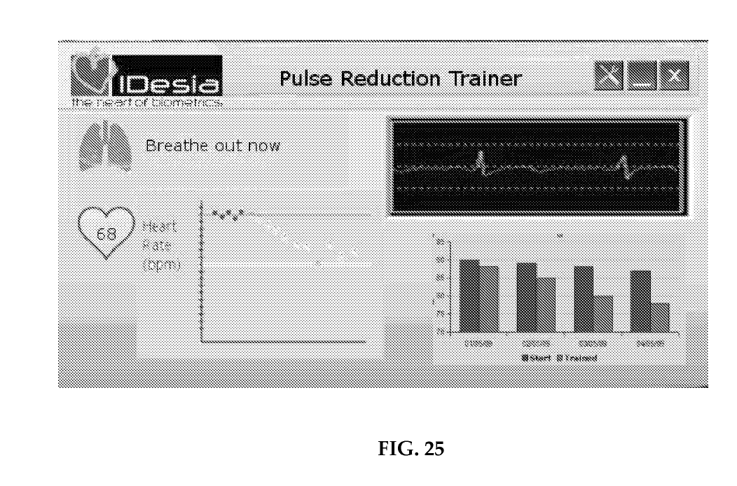

In addition to simple pulse rate reduction feedback, such as shown in the screen shot of FIG. 24, signature analysis of stress state can be used to modify a subject's breathing procedures. For example, high levels of stress can be more quickly reduced, with attendant reduction of pulse, by deeper, more prolonged breathing exercises done with the aid of the Stress Tracker. FIG. 25 shows a possible user interface for this application.

### Second Aspect:

Biometric identification methods benefit from proper determination of an identification threshold. The identification threshold may be derived from correlation analysis between candidate signatures and registered database signatures. The threshold may be determined using a distribution of empirical data to achieve optimal identification performance. Yet a fixed threshold implicitly assumes deterministic signatures and stationary noise, while in practice signatures are variable and noise depends on mostly unpredictable external influences. Therefore, biometric identification methods, including those according to the first aspect, may be adversely affected by signal and noise variations in database and test readings. In general, this would yield decreased correlations for both matches and mismatches.

Thus, according to the second aspect, methods and systems of biometric identification, including those according to the first aspect, may use a dynamic threshold capable of compensating for the effect of signal variations and noise interference. This aspect yields a dynamic, data-dependent identification threshold. In the preferred embodiment, the dynamic threshold is re-calculated in each identification attempt using a statistical approach to normalize the correlation data and thus enable calculation of a quantifiable, statistically significant identification threshold.

The threshold is shown to be resistant to variable signal and noise conditions.

The preferred method according to this second aspect is based on determination of a confidence limit for a correlation-based scoring between a test signature and a set of registered signatures. These ECG signatures can be empirically determined, but they may also be synthetic, in which case there is no need for a background database in the biometric matching process. Synthetic ECG signatures can be created by using random sets of reconstruction coefficients in the PCA-based ECG model. Alternately, reconstruction coefficient sets may be drawn according to a set of rules extracted from the distributions of real-life reconstruction coefficients derived from real subjects.

In any case, a confidence limit describes, with a given degree of statistical confidence, the upper and lower limits for the values in question. A two-tailed limit describes both upper and lower bounds, while a one-tailed limit describes only an upper or a lower cutoff, with the understanding that there is either no lower or no upper limit to the value of the variable. Confidence limits can be determined statistically, in several different ways, if the variable under consideration meets certain statistical criteria appropriate to each statistical method.

Most statistical methods rely on the values of a normally distributed variable, that is, according to the bell-shaped Gaussian distribution. Normally distributed variables have been well characterized statistically, and their statistical limits can be determined in a straightforward manner based on the variable average and variation.

When a variable is not distributed normally, a normalizing transformation may be used to transform the original variable into a new variable which would then be distributed normally, and may thus be used to determine confidence limits. The appropriate mathematical transformation may be determined using statistical considerations, or by empirical examination of a sufficiently large dataset. In order to express the confidence limits in terms of the original variable, a back-transformation is also required.

Signal cross-correlation analysis may be used for the matching procedure. Values range from −1 (absolute negative correlation) through 0 (no correlation) to +1 (absolute positive correlation). Generally, significantly positive correlation indicates a probable true identification, and thus a one-tailed, upper confidence limit should be used to describe the dynamic identification threshold.

By definition, correlations are bounded variables and thus are not normally distributed. A mathematical transformation is necessary to normalize the correlation distribution allowing determination of the upper confidence limit. Alternatively, empirical techniques which do not rely on such transformations may be used.

A preferred method, described more fully below, is particularly appropriate for correlation analysis. It is based on the Fisher Z transformation, which converts correlations into a normally distributed variable.

Another method may use squared correlations. Since raw correlations are not additive, averages or other statistical functions of correlations have no statistical meaning. Squared correlations are additive, but they are also not normally distributed, so that additional transformations would be required. If prior processing of the correlations changes the distribution of their values, additional transformations may be necessary to account for these changes. These additional transformations include, but are not limited to, logarithms, squares, square roots, and transcendental functions.

Still another method would involve a degree of prior empirical testing, preferably where a large number of candidates are correlated to a large database. The likelihood of false identifications would be directly determined by examination of this database, or appropriate transformations could be empirically determined. However, because this method is not dynamic and must be performed prior to real testing, the effects of testing conditions cannot be easily compensated, requiring development of mathematical models for the influence of noise.

The preferred method according to this second aspect, the Fisher-transform method, involves transformation of the correlations between the candidate signature and the registered signatures in order to obtain a distribution of scores that are more nearly normally distributed. As noted above, data that meets assumptions of normality can be used to derive parametric confidence limits.

The Fisher Z transformation was designed to normalize correlations. The transformation may be expressed as follows:

Z_{f}=arctan h(r)

Where Z_{f} is the transformed value, arctan h is the hyperbolic arc tangent function, and r is the correlation. The arctan h should be expressed in radians.

Once all the correlations are transformed, a one-tailed confidence limit for the transformed scores may be determined by taking the mean of all the transformed correlations and the standard deviations of all the transformed correlations, with the exception of the candidate correlation, and calculating:

Confidence limit=tan h(Z_{f mean}+z*sd_{Zf})

where z is the normal distribution ‘z score’, Z_{f mean} is the mean of transformed correlations with the database, and sd_{Zf} is the standard deviation of the transformed correlations with the data base.

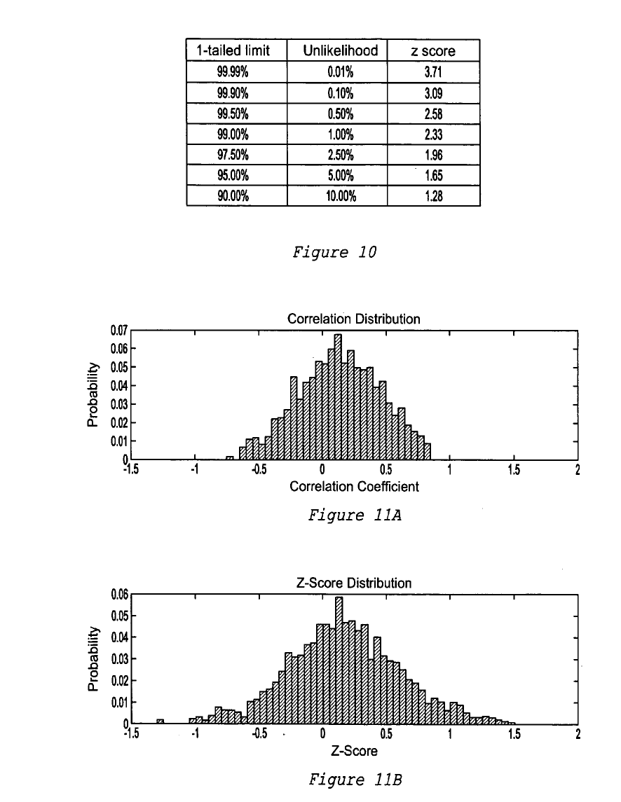

The lower case z here refers to the value of the normal distribution z-score, which is derived based on the desired degree of confidence in the cut-off. A table of such scores is provided in FIG. 10.

In the table of FIG. 10, the standard deviation is multiplied by the appropriate z-score and is added to the mean, and the entire quantity back-transformed to a correlation by taking the hyperbolic tangent.

For example, a 95% confidence limit could be determined using a z score of 1.65. So if the mean of the transformed values was 0.05, and the standard deviation was 0.25, the 95% confidence limit would be 0.72. That is, a correlation value over 0.72 would only occur by chance less than 5% of the time.

A reverse procedure is used to determine the likelihood that any specific candidate identification is due to random chance. By solving for the z-score:

z=(Z_{fc}−Z_{f mean})/Sd_{Zf}

where z is the normal distribution ‘z score’, Z_{fc} is the transformed candidate correlation, Z_{f mean} is the mean of transformed correlations with the database, and sd_{Zf} is the standard deviation of the transformed correlations with the data base.

The resulting z-score can be converted to a 1-tailed probability value by reference to a table of the cumulative normal distribution, and interpolation if necessary. For example, with reference to the abbreviated table above, a z-score of 1.80 would suggest a 3.75% probability that the candidate correlated so highly by chance.

As mentioned above, if noise in the registered signatures or in the candidate signature is random, it would reduce the overall correlations with the candidate value. The true identification, if it exists, would therefore have a lower correlation with the candidate. It should be noted that variability of raw correlations increases as the raw values decrease, since high raw correlations are less variable due to a ceiling effect of maximum correlation of 1, but this is compensated for by the transformation. Thus, a dynamic threshold with the desired certainty may be re-calculated in each identification attempt using the foregoing methods. Importantly, overall random noise still tends to drive all correlations toward zero and reduce overall true variability, thereby lowering the confidence limit accordingly; yet a true match would remain significant as long as the signal to noise ratio does not fall below a certain limit.

The following examples of the second aspect are based on a 38-subject database. All subjects are healthy individuals, participating in the study on a voluntary basis.

### Example 1

### Normalization of Correlations

A set of 703 cross-correlations was obtained by correlating all pairs in the database. The raw and z-transformed correlation distributions are presented in FIG. 11. While raw correlations are not normally distributed (top), the transformed correlations appear to represent a near-normal distribution (bottom).

### Example 2

### Performance

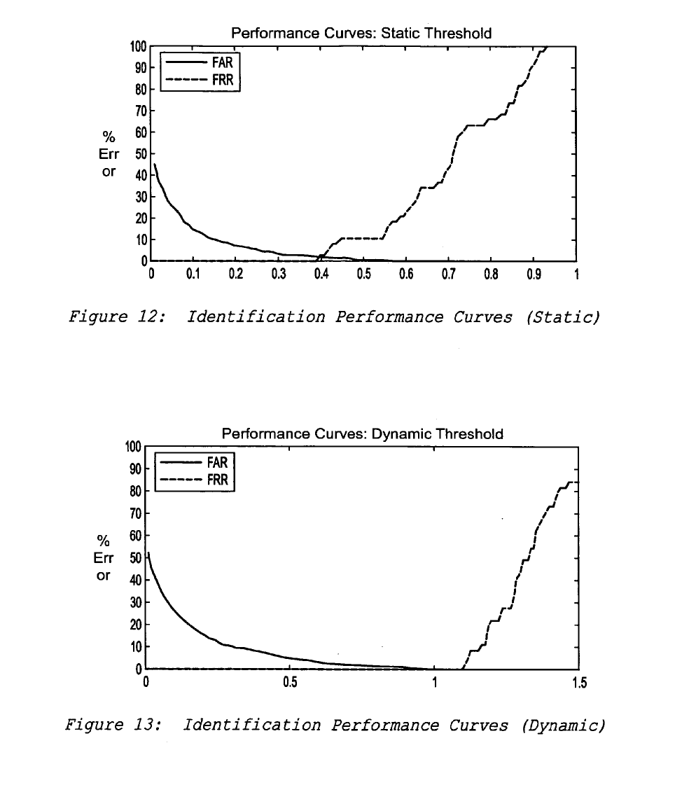

The biometric identification method was implemented using analysis of 38 enrolled signatures and 38 test signatures. FIG. 12 presents FAR and FRR performance curves as a function of a static threshold, and FIG. 13 presents the performance curves as a function of a dynamic threshold. Clearly, the dynamic threshold provides significantly superior results (e.g. EER_{Static}=3%, EER_{Dynamic}=0%).

### Third Aspect:

As described above, the dynamic identification threshold is a data-driven threshold, preferably re-calculated in each identification session to establish a confidence limit and substantiate a statistical significance of the identification process. Yet overall scores still decrease with the drop in signal quality due to background noise, lowering the dynamic threshold and thereby reducing identification confidence. This problem calls for assessment of signal quality in both enrollment and identification phases to facilitate high performance recognition.

The third aspect solves this problem by calculation of a Q value—a type of signal quality index. A quality of signal index Q is a quantitative description of the quality of the ECG signature. It is based on an analysis of the random error in two or more ECG complexes, derived with reference to their signal average ECG.

The Q value may be used to confirm signal quality during the enrollment and identification phases, ensuring adequate system performance. In case of a Q factor lower than required by a predefined threshold (itself based on the desired level of identification confidence) the measurement may either be extended or repeated until the confidence requirement is met.

One preferred methodology derives Q in a series of steps:

(1) The input ECG signal is segmented into ECG complexes comprised of the conventional wave morphology features (e.g. P, Q-R-S, T elements).

(2) The ECG complexes are aligned (“time-locked”) relative to the R wave peak.

(3) An average ECG is derived from the aligned ECG complexes. The preferred method is to take an arithmetic mean, although other methods may be employed, such as the harmonic mean, geometric mean, weighted mean, or median. Other alternatives include transforming the original signals by other methods such as by Principal Component Analysis.

(4) Each original ECG complex is processed relative to the average ECG, such that some difference is derived against the average ECG. The preferred method is to perform subtraction, i.e. original ECG minus average ECG, although other methods may be employed (e.g. division of the original ECG by average ECG). If the average ECG is a stable and true representation of the subject's ECG, then the resulting difference is a representation of the noise inherent in each individual ECG complex (ECG noise).

(5) Each sample point which corresponds in time across each ECG noise complex is processed together to derive a measure of variability. The most preferred method is to determine the variance. Other measures that may be employed include standard deviation or range.

(6) An average is taken of these measures of variability. The most preferred method is to take an arithmetic average. Other methods may involve taking averages after transformation (e.g. log), or taking alternative averages (geometric, harmonic, median). Other summary scores may also be employed, such as the maximum.

Noting that the signal may be normalized prior to analysis, the average may itself be employed as a Q index, as it is directly related to the SNR. Alternatively, various other scaling transformations may be applied to the average to convert it to an index with the desired minima, maxima, and linearity characteristics.

### Example 1 According to the Third Aspect

### Q (Signal Quality) vs. NSR (Noise to Signal Ratio)

If X denotes the ECG data matrix, each row representing one ECG complex may be denoted xᵢ(n) where i is the index of an ECG complex and n represents a discrete time unit. The average of all ECG complexes is denoted x(n). For every point in time n we calculate the error term: eᵢ(n)=xᵢ(n)−x(n), whose variance shall be denoted: σe²(n). A preferred scaling conversion, transforming the average of variability into a zero to one range is defined as follows:

Q=(1+100*σe²(n))^{-0.5}

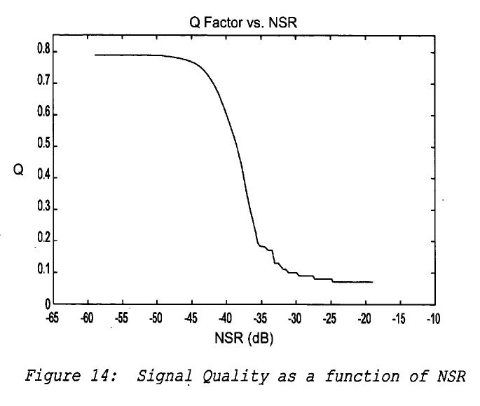

A simulation shown in FIG. 14 demonstrates the utility of using the above Q factor to assess the signal to noise level. This simulation uses real-life ECG recordings with increasing levels of Gaussian white noise added to the signal. FIG. 14 presents Q values as a function of the Noise to Signal Ratio (NSR). It can be seen that once Q starts to decline from its plateau, it drops monotonically with the increase in NSR, until the ECG alignment procedure breaks down (NSR˜−35 dB, Q˜0.2).

### Example 2 According to the Third Aspect

### Score as a Function of Signal Quality

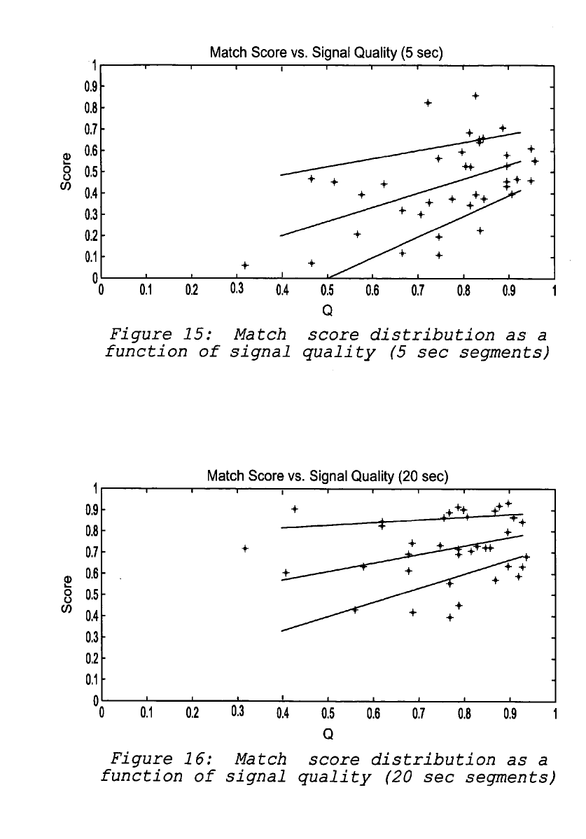

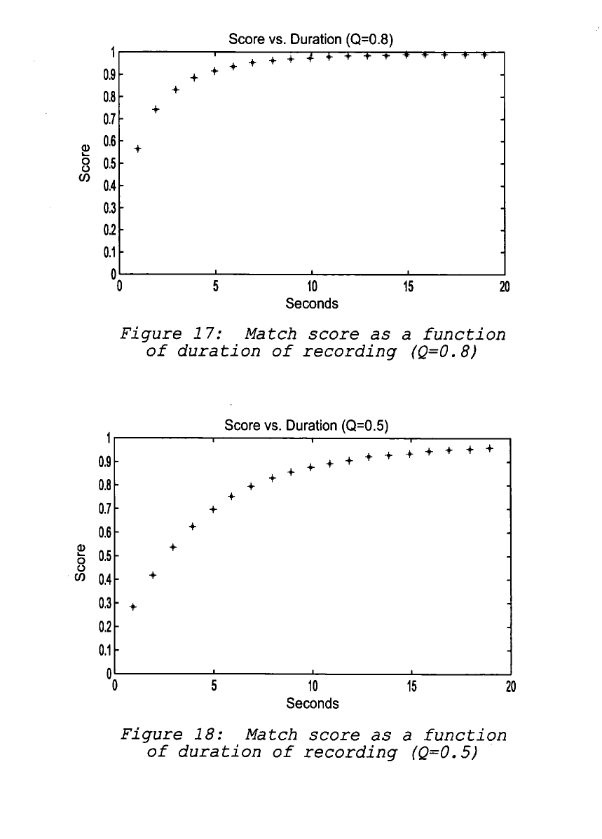

In theory, match scores close to 1 indicate a positive match, while non-match scores should tend to zero indicating complete lack of correlation. In practice, however, true match scores are influenced by temporal variations in the ECG signature and, more significantly, from background noise. Thus, a higher signal quality is required for short time, high scored identification. It should be noted that high quality signal increases the upper bound on match score, but does not influence the lower bound which depends on the cardiologic signature variability. The example represented by FIGS. 13 and 14 demonstrates score distribution as a function of signal quality, based on a database of 38 subjects. FIG. 15 shows short data segments of 5 seconds each. In contrast, FIG. 16 shows longer segments of 20 seconds each (FIG. 16). Obviously, with longer segments the effect of noise is compensated to some extent and the score distribution flattens.

### Example 3 According to the Third Aspect

### Signal Quality and Duration of Recording

Signal quality may be quantified using the Q parameter. With smaller Q values, and provided that Q does not fall below a certain limit where the ECG alignment process breaks down, longer recordings are necessary to maintain a certain level of statistical significance. FIGS. 15 and 16 show the increase in identification score as a function of the length of recording for a given Q value.

Thus, according to this third aspect, the methods and systems disclosed herein may calculate signal quality using a Q-factor or other measure, and cause the system to seek a sample with reduced noise or to take a longer sample based on the Q-factor or other signal quality measure and the desired degree of identification confidence.

### Fourth Aspect:

According to a fourth aspect, the methods and systems disclosed herein may encrypt stored signatures. This safety feature is designed to prevent misuse of the data in the database notwithstanding that the various methods and systems herein typically operate on stored signatures rather than raw ECG data. Thus, an added layer of security may be employed by encrypting the signatures themselves. To that end, a variety of scrambling techniques may be used including the PKI (public key infrastructure) techniques used for credit card data. This fourth aspect makes improper use of the enrolled subject's data all the more difficult, since an unauthorized person would have to decrypt the signature and then still need to convert the signature back into a raw data signal, an impossible task without knowing which common features were removed from the raw data. Thus, one advantage of the systems and methods disclosed herein is that they make it extremely difficult for anyone to misuse the stored information.

### Fifth Aspect:

Biometric identification systems are in general vulnerable to enrollment fraud. The systems and methods according to this fifth aspect solve this problem by using ECG data from genetically related individuals who have enrolled in the database. Immediate family members often have ECGs that share common features. By correlating a subject's signature with the general population and/or with those enrollees he or she is purportedly related to, the system can confidently determine whether or not the subject is who they purport to be. This technique can be used in addition to confirming the individual's identity through conventional methods such as picture identification and/or fingerprint matching. However, unlike those methods, which are non-Euclidian and not amenable to clustering based on similarity, this technique can determine fraud at any stage of enrollment process by determining a probability of a genetic relationship based on the enrollee's ECG signature.

### Sixth Aspect:

The systems and methods disclosed herein may also make use of ultra-high impedance probes to measure ECG. Since reliability and ease of use is important for an ECG-based biometric identification system, it is advantageous to measure an ECG at a single point, or even without touching the subject. Electric potential probes can work with biometric methods and systems, including those described herein, to increase reliability and ease of use for biometric identification. Ultra-high impedance probes come in a variety of forms. See e.g. Electric potential probes—new directions in the remote sensing of the human body, Harland et al., Meas. Sci. Technol. 13 (2002) 163-169. The ultra-high input impedance probes according to this aspect preferably have ultra-low noise characteristics, and do not require a current conducting path in order to operate. As a result, they work well with the foregoing methods and systems even when used by a layperson without the help of an expert system operator. Thus, these probes may be used in airport-based biometric identification systems, such as by acquiring an ECG signal when an individual passes through a scanner (similar to a metal detector) in full dress. Likewise, a single probe may be used to collect an ECG from an individual's finger tip, such as at an ATM or gaming machine. The use of a single probe contact gives the subject more freedom of movement and makes it easier for him or her to comply with the identification and enrollment regimen. This is particularly useful when the biometric identification systems described herein are used to control the subject's operation of machinery, especially when the machine requires physical contact to operate (e.g., a firearm or vehicle). The single probe and remote probe ECG capture systems according to this aspect may also be complemented by noise reduction strategies to reduce body noise and EMG.

### Seventh Aspect:

According to a seventh aspect, a biometric identification method and system may correlate the match scores for a subject (which are created by comparing the subject's signature with those of database enrollees) with the match scores of a plurality of enrollees (which are created by comparing the enrollees' signatures with those of database enrollees). Thus, rather than analyzing a distribution of a subject's correlated match scores, this identification technique analyzes the distribution of the correlation of a subject's match scores and those of the enrollees. As with the fifth aspect, the methods and systems according to this aspect are useful for identifying related individuals. This is because an individual related to a group of enrollees will have a Gaussian distribution of match scores that has a substantially higher median than a Gaussian distribution of the match scores for an individual unrelated to the enrollees. Thus, by examining the distribution of match scores, the probability of a subject's genetic relationship with the enrollees may be confirmed.

### Eighth Aspect:

Finally, in the alternative or in addition to the correlation techniques described above, the methods and systems described herein may employ a weighted correlation for identification. According to this aspect, the correlation may give different weights to various signature differences. For example, signature differences due to QRS complex features may be weighted more than signature differences due to T or P complex features. The systems and methods may also use the root mean square of the signature values as part of a weighting function since T is highly variable, QRS is stable, and P is somewhere in the middle. Thus, the signatures may be normalized using root-mean-square computations, L1 metrics or another normalizing technique.

Preferred Embodiment that may be Used with All Aspects:

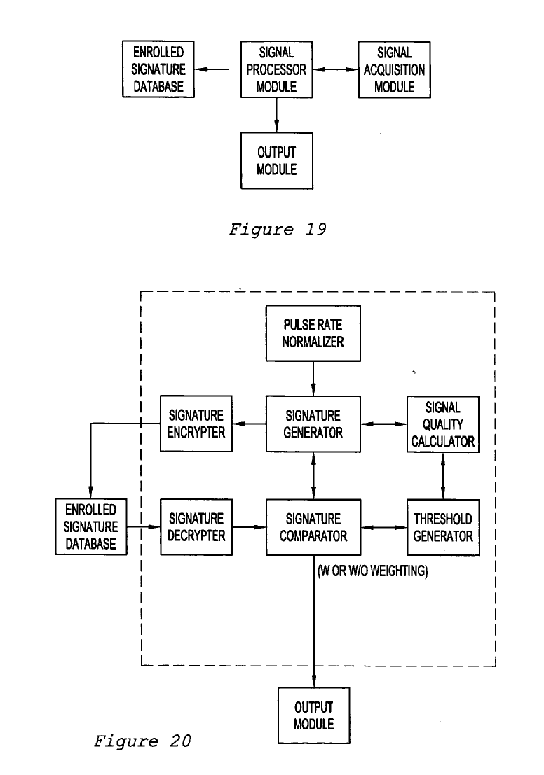

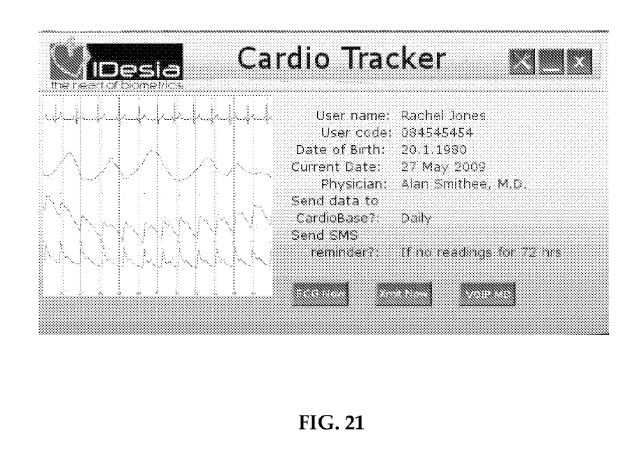

FIG. 19 shows a functional diagram of a preferred system. Likewise, FIG. 20 shows a functional diagram of a preferred signal processor. The term “processor” is used herein generically and the processing may be done by physically discrete components, such as with co-processors on an IC chip, or the processor may comprise a physically integral unit.

General Example that may be Used with All Aspects: Enrollment Algorithm

The following is an example algorithm for an enrollment phase that may be used with any of the foregoing aspects:
i. Let xᵢ(n) represent a 20-second, 250 Hz digitized sample of the i^{th} new subject, where n denotes discrete units of time.
ii. xᵢ(n) is band-pass filtered in the range 4 Hz-40 Hz.
iii. The filtered signal is denoted yᵢ(n)
iv. The filtered signal yᵢ(n) is searched for QRS complexes, identifying the ‘R’ peaks as anchor points.
v. The filtered signal yᵢ(n) is maintained or inverted to obtain positive ‘R’ peaks.
vi. The identified QRS complexes are counted to establish an average pulse rate reading PRᵢ.
vii. The filtered signal yᵢ(n) is segmented around the anchor points, taking 50 samples before and 90 samples after each ‘R’ anchor point.
viii. Each data segment is normalized by the amplitude of the ‘R’ anchor point.
ix. The segments are aligned around the anchor points and averaged to produce the subject electro-cardiologic signal, denoted sᵢ(n).
x. The subject electro-cardiologic signal sᵢ(n) is adjusted according to the average pulse rate PRᵢ, by normalizing ‘P’ and ‘T’ latencies according to the pulse rate. The adjusted electro-cardiologic signal is denoted υᵢ(n).
xi. The pulse rate adjusted subject's electro-cardiologic signal υᵢ(n) is added to the database and is introduced into a grand-average T(n).
xii. A set of electro-biometric signatures Φᵢ is constructed by subtraction of the grand-average T(n) from each of the pulse rate adjusted electro-cardiologic signals stored in the system database.

### Example

### Recognition Algorithm

The following is an example an algorithm for the recognition phase:
i. Let xⱼ(n) represent a 10-second, 250 Hz digitized sample of the tested subject.
ii. xⱼ(n) is band-pass filtered in the range 4 Hz-40 Hz.
iii. The filtered signal is denoted yᵢ(n)
iv. The filtered signal yᵢ(n) is searched for the locations of QRS complexes, using the R peak as an anchor point.
v. The filtered signal yᵢ(n) is maintained or inverted to obtain positive ‘R’ peaks.
vi. The identified QRS complexes are counted to establish an average pulse rate reading PRⱼ.
vii. The filtered signal yⱼ(n) is segmented around the anchor points, taking 50 samples before and 90 samples after each anchor point.
viii. The segments are aligned around the anchor points and averaged to produce the subject electro-cardiologic signal, denoted sⱼ(n).
ix. The subject electro-cardiologic signal sⱼ(n) is normalized according to the average pulse rate PRⱼ. The pulse rate adjusted subject electro-cardiologic signal is denoted υⱼ(n).
x. An electro-biometric signature σⱼ is constructed by subtraction of the grand-average T(n) from the pulse rate adjusted electro-cardiologic signal υᵢ(n).
xi. The correlation coefficients between the electro-biometric signature σⱼ and all the enrolled electro-biometric signatures Φᵢ are calculated and squared, maintaining their original arithmetic sign.
xii. The largest sign-maintained squared correlation value is selected and compared to a preset threshold.
xiii. If the selected largest sign maintained squared correlation value is larger than the preset threshold then a positive match is indicated, and the subject is identified.

Thus, a method and apparatus of acquisition, processing, and analysis of electro-cardiologic signals for electro-biometric identity recognition may include any subset of the following enrollment and recognition steps:

### Enrollment

Acquisition, digitization, and storage of electro-cardiologic signals from subjects;
a. Formation of an electro-cardiologic signal database;
b. Partition of the template database into several subsets based on electro-cardiologic signal similarity;
c. Construction of one or more grand averages;
d. Derivation of subject-specific electro-biometric signatures.

### Recognition

### Verification

The newly captured electro-biometric signature is compared with the subject specific enrolled electro-biometric signature template;
a. Correlation and confidence analysis of the newly captured subject electro-biometric signature with the relevant stored electro-biometric signature template;
b. Display and registration of the recognition result and/or activation of a physical or virtual local/remote mechanism.

### Identification

The newly captured electro-biometric signature is compared with all of the electro-biometric signature templates participating in the database;
a. Correlation and confidence analysis of the newly captured subject electro-biometric signature with all stored electro-biometric signature templates;
b. Display and registration of the recognition result and/or activation of a physical or virtual local/remote mechanism.

In a preferred embodiment, the E-BioID system measures an electrical bio-signal from the human body through conductive sensor plates. These same plates may be used for bidirectional interaction with the subject's nervous system, for example, by inducing a sympathetic skin response in the user with small magnitude electrical stimulation that is provided through the plates. Such bidirectional interaction constitutes a biological challenge-response mechanism that ensures submission of a fresh bio-signal without requiring active participation of the user in the challenge-response procedure.

It is noteworthy that various modules and engines may be located in different places in various embodiments.

Others may readily modify and/or adapt the embodiments herein for various applications without undue experimentation and without departing from the generic concept. Such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms and still fall within the literal or equivalent scope of the claims.

Thus the expressions “means to . . . ” and “means for . . . ”, or any method step language, as may be found in the specification above and/or in the claims below, followed by a functional statement, are intended to define and cover whatever structural, physical, chemical or electrical element or structure, or whatever method step, which may now or in the future exist which carries out the recited function, whether or not precisely equivalent to the embodiment or embodiments disclosed in the specification above, i.e., other means or steps for carrying out the same functions can be used; and it is intended that such expressions be given their broadest interpretation.

An exemplary computing system may be used to implement various embodiments of the systems and methods disclosed herein. The computing system may include one or more processors and memory. The memory may include a computer-readable storage medium. Common forms of computer-readable storage media include, for example, a floppy disk, a flexible disk, a hard disk, magnetic tape, any other magnetic medium, a CD-ROM disk, digital video disc (DVD), various forms of volatile memory, non-volatile memory that can be electrically erased and rewritten. Examples of such non-volatile memory include NAND flash and NOR flash and any other optical medium, the memory is described in the context of. The memory can also comprise various other memory technologies as they become available in the future.

Main memory stores, in part, instructions and data for execution by a processor to cause the computing system to control the operation of the various elements in the systems described herein to provide the functionality of certain embodiments. Main memory may include a number of memories including a main random access memory (RAM) for storage of instructions and data during program execution and a read only memory (ROM) in which fixed instructions are stored. Main memory may store executable code when in operation. The system further may include a mass storage device, portable storage medium drive(s), output devices, user input devices, a graphics display, and peripheral devices. The components may be connected via a single bus. Alternatively, the components may be connected via multiple buses. The components may be connected through one or more data transport means. Processor unit and main memory may be connected via a local microprocessor bus, and the mass storage device, peripheral device(s), portable storage device, and display system may be connected via one or more input/output (I/O) buses. Mass storage device, which may be implemented with a magnetic disk drive or an optical disk drive, may be a non-volatile storage device for storing data and instructions for use by the processor unit. Mass storage device may store the system software for implementing various embodiments of the disclosed systems and methods for purposes of loading that software into the main memory. Portable storage devices may operate in conjunction with a portable non-volatile storage medium, such as a floppy disk, compact disk or Digital video disc, to input and output data and code to and from the computing system. The system software for implementing various embodiments of the systems and methods disclosed herein may be stored on such a portable medium and input to the computing system via the portable storage device. Input devices may provide a portion of a user interface. Input devices may include an alpha-numeric keypad, such as a keyboard, for inputting alpha-numeric and other information, or a pointing device, such as a mouse, a trackball, stylus, or cursor direction keys. In general, the term input device is intended to include all possible types of devices and ways to input information into the computing system. Additionally, the system may include output devices. Suitable output devices include speakers, printers, network interfaces, and monitors. Display system may include a liquid crystal display (LCD) or other suitable display device. Display system may receive textual and graphical information, and processes the information for output to the display device. In general, use of the term output device is intended to include all possible types of devices and ways to output information from the computing system to the user or to another machine or computing system. Peripherals may include any type of computer support device to add additional functionality to the computing system. Peripheral device(s) may include a modem or a router or other type of component to provide an interface to a communication network. The communication network may comprise many interconnected computing systems and communication links. The communication links may be wireline links, optical links, wireless links, or any other mechanisms for communication of information. The components contained in the computing system may be those typically found in computing systems that may be suitable for use with embodiments of the systems and methods disclosed herein and are intended to represent a broad category of such computing components that are well known in the art. Thus, the computing system may be a personal computer, hand held computing device, telephone, mobile computing device, workstation, server, minicomputer, mainframe computer, or any other computing device. The computer may also include different bus configurations, networked platforms, multi-processor platforms, etc. Various operating systems may be used including Unix, Linux, Windows, Macintosh OS, Palm OS, and other suitable operating systems. Due to the ever changing nature of computers and networks, the description of the computing system is intended only as a specific example for purposes of describing embodiments. Many other configurations of the computing system are possible having more or less components.

While various embodiments have been described above, it should be understood that they have been presented by way of example only, and not limitation. The descriptions are not intended to limit the scope of the invention to the particular forms set forth herein. Thus, the breadth and scope of a preferred embodiment should not be limited by any of the above-described exemplary embodiments. It should be understood that the above description is illustrative and not restrictive. To the contrary, the present descriptions are intended to cover such alternatives, modifications, and equivalents as may be included within the spirit and scope of the invention as defined by the appended claims and otherwise appreciated by one of ordinary skill in the art. The scope of the invention should, therefore, be determined not with reference to the above description, but instead should be determined with reference to the appended claims along with their full scope of equivalents.

What is claimed is:

1. A method for tracking changes in an electro-cardiologic signal using a signal processing device having a sensor, the method comprising:
communicatively coupling the sensor of the signal processing device with a subject;
reading into a memory an electro-cardiologic signal of the subject via the sensor and the signal processing device to create an enrolled electro-cardiologic signal;
tracking, via the signal processing device, possible variations in the electro-cardiologic signal of the subject over a time period to modify the enrolled electro-cardiologic signal; and
adjusting the enrolled electro-cardiologic signal when changes in the electro-cardiologic signal occur.

2. The method of claim 1, wherein the signal processing device is worn on a subject's body.

3. The method of claim 1, wherein the signal processing device is worn on a wrist of the subject.

4. The method of claim 3, wherein the signal processing device is located within a watch.

5. The method of claim 1, further comprising creating an individualized biometric signature of the subject from the electro-cardiologic signal of the subject.

6. The method of claim 5, further comprising monitoring a variation in the individualized biometric signature of the subject, wherein the biometric signature varies based on relative physiological states of the subject.

7. The method of claim 6, further comprising measuring and examining changes in the biometric signature of the subject.

8. The method of claim 7, wherein the measuring and examining are performed using an individualized historical database.

9. The method of claim 7, wherein the changes in the biometric signature relate to a physiological state of the subject.

10. The method of claim 7, wherein the changes in the biometric signature relate to a physiological mood of the subject.

11. The method of claim 7, wherein the changes in the biometric signature relate to a pulse rate of the subject

12. The method of claim 7, wherein the changes in the biometric signature relate to physiological stress of the subject.

13. A method for monitoring a subject, the method comprising:
tracking changes in an electro-cardiologic signal using a signal processing device having a sensor; and
simultaneously performing biometric signature identification.

14. The method of claim 13, wherein performing biometric signature identification is initiated subsequently in time to commencement of tracking changes in the electro-cardiologic signal.

15. The method of claim 13, wherein tracking changes in the electro-cardiologic signal is initiated subsequently in time to commencement of performing biometric signature identification.

16. The method of claim 13, wherein tracking changes in an electro-cardiologic signal comprises:
communicatively coupling the sensor of the signal processing device with the subject;
reading into a memory an electro-cardiologic signal of the subject via the sensor and the signal processing device to create an enrolled electro-cardiologic signal;
tracking, via the signal processing device, possible variations in the electro-cardiologic signal of the subject over a time period to modify the enrolled electro-cardiologic signal; and
adjusting the enrolled electro-cardiologic signal when changes in the electro-cardiologic signal occur.

17. The method of claim 13, wherein performing biometric signature identification comprises:
producing and storing a first biometric signature that identifies a specific subject by forming the difference between a representation of a heartbeat pattern of the specific subject and a stored representation of common features of heartbeat patterns of a plurality of subjects;
after said producing step, obtaining a representation of a heartbeat pattern of a selected subject and producing a second biometric signature by forming the difference between the heartbeat pattern of the selected individual and the stored representation of common features of the heartbeat patterns of the plurality of individuals; and
comparing the second biometric signature with the first biometric signature to determine whether the selected subject is the specific subject.

18. An electronic device operative to detect a subject's electro-cardiologic signal, the device comprising:
a housing operative to detect an electro-cardiologic signal of the subject; and
a signal processing module communicatively coupled with the housing and operative to receive and process the detected electro-cardiologic signal.

19. The method of claim 18, wherein the housing functions as a heart sensor.

20. The method of claim 18, wherein a first portion of the housing functions as a first heart sensor.

21. The method of claim 18, wherein a second portion of the housing functions as a second heart sensor.

22. The electronic device of claim 18, wherein the housing is formed from a conductive medium in contact with the subject, the housing transmitting the subject's electro-cardiologic signal to a signal acquisition module communicatively coupled with the signal processing module.

23. The electronic device of claim 18, further comprising an output module communicatively coupled with the signal processing module.

24. A method for tracking changes in an electro-cardiologic signal using a signal processing device having a sensor, the method comprising:
communicatively coupling the sensor of the signal processing device with a subject;
reading into a memory an electro-cardiologic signal of the subject via the sensor and the signal processing device to create an enrolled electro-cardiologic signal;
tracking, via the signal processing device, possible variations in the electro-cardiologic signal of the subject over a time period to modify the enrolled electro-cardiologic signal; and
adjusting the enrolled electro-cardiologic signal when changes in the electro-cardiologic signal occur.

25. A method for playing a game by tracking changes in an electro-cardiologic signal using a signal processing device having a sensor, the method comprising:
communicatively coupling the sensor of the signal processing device with a subject;
reading into a memory an electro-cardiologic signal of the subject via the sensor and the signal processing device to create an enrolled electro-cardiologic signal;
tracking, via the signal processing device, possible variations in the electro-cardiologic signal of the subject over a given time period to detect changes in a physiological state of the subject; and
providing output indicia of at least one positive outcome in the game for the subject when at least one variation in the electro-cardiologic signal of the subject over the given time period exceed one or more threshold values.

26. A computer readable storage medium having a program embodied thereon, the program executable by a processor to perform a method for tracking changes in an electro-cardiologic signal using a signal processing device having a sensor, the method comprising:
communicatively coupling the sensor of the signal processing device with a subject;
reading into a memory an electro-cardiologic signal of the subject via the sensor and the signal processing device to create an enrolled electro-cardiologic signal;
tracking, via the signal processing device, possible variations in the electro-cardiologic signal of the subject over a time period to modify the enrolled electro-cardiologic signal; and
adjusting the enrolled electro-cardiologic signal when changes in the electro-cardiologic signal occur.
